(19) United States
(12) Patent Application Publication
(10) Pub. No.: US 2009/0227954 A1
(43) Pub. Date: Sep. 10, 2009

(54) Multisegment Interconnect Device for Elastic Tubing

(76) Inventors: David A. Loiterman, Oak Brook, IL (US); Michael G. Loiterman, Yorkville, IL (US)

Correspondence Address: VEDDER PRICE P.C., 222 N. LASALLE STREET, CHICAGO, IL, 60601, US

(21) Appl. No.: 12/399,772

(22) Filed: Mar. 6, 2009

Related U.S. Application Data
(60) Provisional application No. 61/034,317, filed on Mar. 6, 2008.

(51) Int. Cl. A61M 25/16; F16L 35/00

(52) U.S. Cl. 604/175; 285/332; 604/535

(57) ABSTRACT

The present disclosure generally relates to an multisegment interconnect device for permanently connecting adjacent elastic tubes, catheter ends, or other fluid transportation conduits where tubes are push-locked over conical outer ends each with a locking lip, the segments are connected at an interface, and the segments are adapted in length or geometry to permanently connect the tubes and hold a different length of tube over the body of the multisegment interconnect device.

### CROSS-REFERENCE TO RELATED APPLICATION

The present patent application claims priority from and the benefit of U.S. Provisional Patent Application No. 61/034,317, filed Mar. 6, 2008, and entitled Interconnect Device for Elastic Tubing, which prior application is hereby incorporated herein by reference.

### FIELD OF THE DISCLOSURE

The present disclosure generally relates to a multisegment interconnect device permanently connecting adjacent elastic tubes, catheter ends, or other transportation conduits. More specifically, the present disclosure generally relates to a multisegment interconnect device with a push-lock device with where tubes are locked over locking lips and the interconnect can be adapted using an interface to different lengths or geometries.

### BACKGROUND

Catheters and other polymer-based or flexible tubes are used during medical interventions in a wide variety of biomechanical applications. Medical treatment relies on highly specialized devices adapted to meet unique needs associated with precise and constantly evolving procedures. Because medical mistakes often result in health consequences, long-term disability, or even death, devices used in this field, while often derived from other well-known fields, must be specifically designed with unique biomedical requirements in mind. For example, medical saws used to cut bone, while derived from carpentry devices, are adapted for doctors in many important ways.

Some tubes are designed to be fully or partly implanted in the human body. One type of tubing is the medical catheter, which is a small, hollow tube introduced within a patient's body to extract bodily fluids from the body, circulate fluid from the body to an external piece of equipment, or even provide access to the internal body elements. For example, some catheters can be used during the dialysis process where an external device filters a plurality of chemicals and compounds such as urea from blood, or an external device adjusts the volume of said fluid in a patient. A catheter may also be inserted in the urethra when the conduit is damaged, in the abdomen in the case of an abdominal abscess, for the administration of intravenous fluids during angioplasty, angiography, or balloon septostomy, for administration of anesthetic medication, or for the subcutaneous administration of insulin or other medication for medical treatment such as chemotherapy.

Catheters or other tubes inserted in the body are often damaged when they are connected and disconnected from equipment. These tubes are also cut by physicians to specific lengths adapted to their application. As medical conditions evolve or change, the lengths of these tubes must be adapted precisely. Devices placed within a body can also be provided with a fixed length of tubing that requires adjustment at a later stage of placement. For example, if a patient gains weight, a catheter adapted to reach the outer layer of skin may require an adjustment in length. Accordingly, physicians require tools designed to shorten, lengthen, or connect with precision different catheters and tubes.

FIGS. 1 and 2 are taken from U.S. Pat. No. 4,537,183 directed to a connector device that is placed subcutaneously in the groin area as part of a penile prosthesis. This connector includes a rigid connector component over which end tubing is pushed until it reaches a middle portion. A stop is placed in the middle portion of the body and since the body of this device is not designed to hold the tube in place over the rigid connector, two locking clips are pushed inwardly until they lock the tube in place over the middle portion of the rigid connector.

This prior art device requires the use of locking clips. If these clips are moved out of position, for example after use or if the system is jarred, they can disengage or leak. The locking clips also increase the weight, size, and cost of the device. Since this connector is placed in the groin area, a shock such as a kick can result in the unlocking of the device leading to surgical intervention to reconnect the tubes to the connector. This prior art device provides for the removal of the rigid connector from the tube endings, and ultimately, reuse of the end portions of connecting tubes. Such a capacity for disconnection may be desirable by the physician in some cases but may also not be in other obvious conditions of operation.

What is needed is a connector capable of quickly and permanently joining tubes, where the connector can be quickly adapted for placement in different configurations.

### SUMMARY

The present disclosure generally relates to an multisegment interconnect device for permanently connecting adjacent elastic tubes, catheter ends, or other transportation conduits where tubes are push-locked over conical outer, ends each with a locking lip, the segments are connected at an interface, and the segments are adapted in length or geometry to permanently connect the tubes and hold a different length of tube over the body of the multisegment interconnect device. This disclosure also relates to methods of modifying an elastic tube connection at the above multisegment interconnect device and methods of modifying the length of an elastic tube equipped with the above multisegment interconnect device.

### BRIEF DESCRIPTION OF THE DRAWINGS

Certain embodiments are shown in the drawings. However, it is understood that the present disclosure is not limited to the arrangements and instrumentality shown in the attached drawings.

FIG. 1 taken from the prior art is an elevation view illustrating the method of attaching a tubing with a connector in an implanted prosthesis device.

FIG. 2 is an enlarged cross-sectional view of the connector of FIG. 1 from the prior art.

FIG. 3 is a front sectional view of an interconnect device for elastic tubing in a short configuration according to one contemplated embodiment of the present disclosure.

FIG. 4 is an elevation view illustrating the method of attaching elastic tubing with the connector shown in FIG. 3 according to an embodiment of the present disclosure.

FIG. 5 is a side view of the device shown as FIG. 3 along lines 5-5.

FIG. 6 is a front section view of an interconnect device for elastic tubing in a long configuration according to another embodiment of the present disclosure in a working configuration.

FIG. 7 is a front section view of a single-piece interconnect device as shown in FIG. 6 according to another embodiment of the present disclosure.

FIG. 8 is an elevation view illustrating an interconnect device with a different male adaptor according to another embodiment of the present disclosure.

FIG. 9 is a diagrammatic representation of a method of modifying an elastic tube connection at a connector where elastic tubes are permanently affixed to each of the opposite connecting ends of a multisegment interconnect device.

FIG. 10 is a diagrammatic representation of methods for modifying the length of an elastic tube by changing a segment of a multisegment interconnect device.

FIG. 11 is a diagrammatic representation of a method for connecting an internal catheter equipped with a multisegment interconnect device to an implanted port or an external catheter.

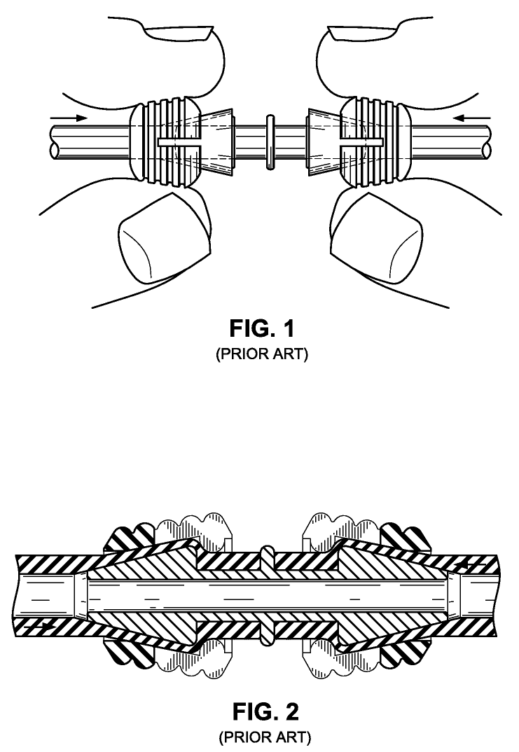

### DETAILED DESCRIPTION OF THE INVENTION

For the purposes of promoting and understanding the principles disclosed herein, reference is now made to the preferred embodiments illustrated in the drawings, and specific language is used to describe the same. It is nevertheless understood that no limitation of the scope of the invention is hereby intended. Such alterations and further modifications in the illustrated devices and such further applications of the principles disclosed and illustrated herein are contemplated as would normally occur to one skilled in the art to which this disclosure relates.

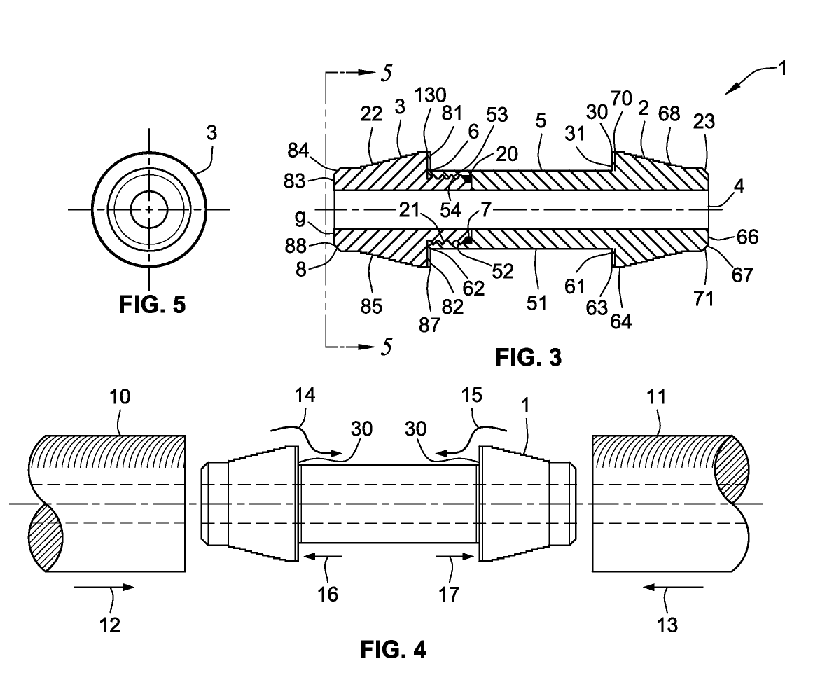

The ending of subcutaneous devices, when in contact with one of the fluid systems of the human body, such as blood, is connected via the interconnect device as defined hereafter and shown in FIGS. 3-8 to either a subcutaneous device such as a port for recurring dialysis treatments, a tube, and the like, or to external vascular access interfaces or devices or any external device or tube. For example, a catheter may be used in the case of chemotherapy or dialysis treatments that result from a temporary or extraordinary condition that does not warrant or allow the use of an implanted subcutaneous device. The interconnect device then connects at one end to the catheter and at the other end to any and all types of interfaces found in the medical field or tubes.

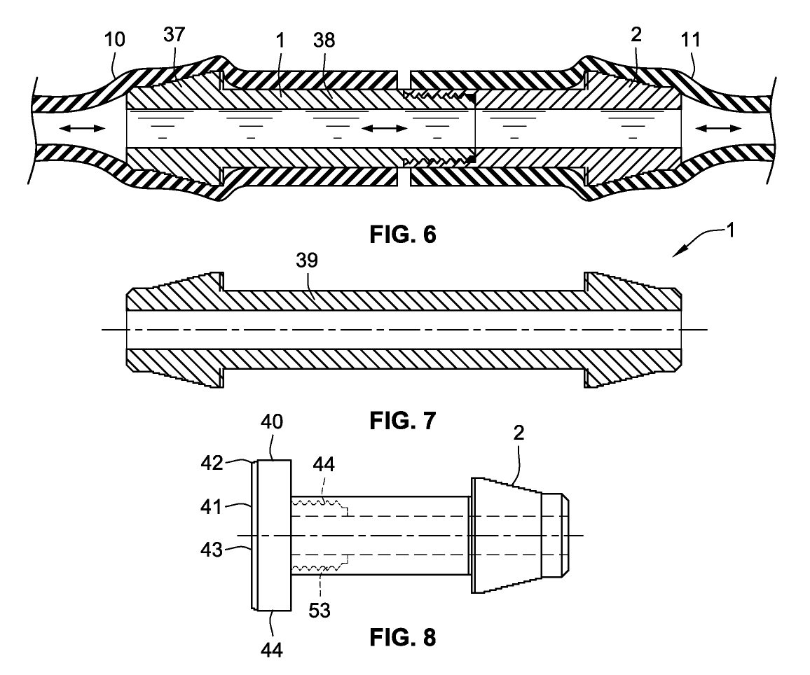

FIG. 3 is a front sectional view of an interconnect device 1 for elastic tubing in a short configuration according to one contemplated embodiment of the present disclosure. FIG. 4 is an elevation view illustrating the method of attaching elastic tubing with the connector shown in FIG. 3 according to an embodiment of the present disclosure. Tubes 12, 13 are slipped 14, 15 over locking lips 30 until they joint at a midportion of the body of the device 1. FIG. 6 shows a long configuration of the device 1 where the tubes 12, 13 cover the greater portion of the body.

FIG. 3 shows a front sectional view of an interconnect device 1 for connection of two opposing elastic tubes 10, 11. The tubes 10, 11 have an internal opening through which fluid such as blood passes. When the tubes 10, 11 connect the interconnect device 1, their openings align with a middle passage 4 made in the device 1 that allows fluid to pass from a first tube through the device 1 and out the second tube 12, 13 as shown in FIG. 4.

The interconnect device 1 as shown is symmetrical, which allows connection of tubes 12, 13 with similar internal and external diameters and with internal diameters of approximately the same internal diameter of the middle passage 4. The use of nonsymmetrical devices 1 where one end is larger in size to accommodate a larger or thicker tube, or even a curved or angled device to accommodate a connection that is not along a single axis, is also contemplated. In one preferred embodiment, the interconnect device 1 is made of titanium alloys of grade Ti-6Al-4V ELI ASTM F-136. While one type of biocompatible alloy is provided, the use of any rigid, semirigid, or flexible material capable of holding the tubes 10, 11 is contemplated.

FIG. 7 shows a configuration where the interconnect device 1 is made of a single rigid connector 39. FIGS. 3, 6, and 8 show a configuration where the interconnect device 1 is made of two connecting parts. In FIG. 3, a long female connector 2 is connected to a short male connector 3, whereas in FIG. 6, the long female connector 2 is connected to a long male connector 37, and in FIG. 8, the long female connector 2 is connected to a short end connector 40. Each end connector is connected at an interface by a threaded section mated to each opposite connector in the embodiment shown. A seal 20 is shown in FIG. 3 on the interface 7 between the mating sections of the connectors. While one type of mating or connection is shown that enables the two opposite connectors to be connected into the interconnect device 1, the use of any connecting system, such as clips, locks, tabs, screws, magnets, hooks, or the like that allows for the unobstructed passage of the fluid in the middle passage 4 without creating a leak between the middle passage 4 and the external surface of the interconnect device 1 is contemplated.

In the disclosed system, the tubes 10, 11 as shown in FIG. 4 are slid 12, 13 over a conical portion 22 with a locking lip 30 having an angle able to lock in place the deformable tubes 10, 11. The use of any other equivalent system where the tubes 10, 11 are inserted over the end portions of the interconnect device 1, such system including but not limited to adhesives, thermo or ultraviolet chemical processes, deformable tubes, and the like, is also contemplated. The locking lip 30 as shown has a right angle and is immediately connected to the upper portion of the conical portion 22. The back end 31 of the conical portion 22 as shown is perpendicular to the body 5 of the interconnect device 1. If the lip 30 must be made more pronounced to retain tubes 10, 11 made of a more rigid material, then the back end 31 may be angled inwardly to form an arrowhead.

In the short version shown in FIGS. 3 and 8, the body 5 has an external diameter of 0.115 inch and a length of 0.25 inch. In the long version shown in FIGS. 6 and 7, the external diameter of the body 5 remains 0.115 inch, but the length is increased to 0.50 to allow for a cover zone of the tubes 10, 11 over the body 5 of 0.25 inch per tube. In the preferred embodiment, the conical portion 22 has a length of 0.108 inch and a rim 23 of 0.033 inch in length with a break edge of 0.005 inch at 45 degree in chamfer. The locking lip 30 in one preferred embodiment has a radius of 0.003 inch. While one set of dimensions is provided, the use of other functional dimensions is also contemplated.

FIG. 4 also shows with arrows 14, 15 how the tubes 10, 11 slide over the locking lip 30 of the interconnect device 1 and over the body 5. Once the tubes 10, 11 are pulled back, the inner portion of the tubes 10, 11 lock as shown by arrows 16, 17 against the locking lip 30. In the system shown, the tubes 10, 11 must be cut open or partly removed from the body 5 and the locking lip 30 area.

Finally, in the embodiment shown in FIG. 8, one of the two ends of the interconnect device 1 can include a short end connector 40, such as a connector that can fit into different devices or machines such as a subcutaneous port. In one embodiment, the interconnect device 1 includes an inner passage 4 for connection to elastic tubes 10, 11, each with a central passage shown by the dashed lines in FIG. 4, which is in fluidic communication with the inner passage. The device 1 includes a body 5 with a first end 61, a second end 62 in opposition, a first end segment 2 comprising a first connection surface 63 with an outer edge 64 with a first locking lip 30, a first end surface 66 with a first external periphery 67, and a first conical surface 68 with a large opening 70 and a small opening 71 connected at the large opening 70 to the first locking lip 30 and connected at the first external periphery 67 to the small opening 71.

As shown in FIGS. 3-8, the external radius of the large opening 70 is greater than the external radius of the small opening 71. The device 1 also includes a second end segment 3 comprising a second connection surface 81 with an outer edge 82 with a second locking lip 130, a second end surface 83 with a second external periphery 84, and a second conical surface 85 with a large opening 87 and a small opening 88 connected at the large opening 87 to the second locking lip 130 and connected at the second external periphery 84 to the small opening 88, wherein the external radius of the large opening 87 is greater than the external radius of the small opening 88.

In one embodiment, a multisegment interconnect device 1 has opposing connecting ends 2, 3, described as the long female connector and the short male connector in FIG. 3, for the permanent connection of elastic tubes 11, 12 at the connecting ends 2, 3. The device 1 includes a first end segment 2 with a first body portion 51 with a first interface 53 and a first connecting end 55 to connect to a first elastic tube 13 having a first end surface 66 adjacent to the first connection surface 63 to guide the first tube 13 over the first end surface 66 to the first connection surface 63. The first connection surface 63 forms a first acute outer edge with a first locking lip 30, the first end surface 66 comprises a first external periphery 67, and a first conical surface 68 defining a large opening 70 and a small opening 71 at each end thereof and where the large opening 70 connects to the first acute outer edge.

The device 1 further includes a second end segment 3 shown in FIG. 3 as the short male connector with a second body portion 52 with a second interface 54, and a second connecting end 56 to connect to a second elastic tube 11 having a second end surface 83 adjacent to the second connection surface 81 to guide the second tube 11 over the second end surface 83 to the second connection surface 81. The second connection surface 81 forms an outer acute edge with a second locking lip 130, the second end surface 83 comprises a second external periphery 84, and a second conical surface 85 defining a large opening 87 and a small opening 88 and wherein the large opening 87 connects to the second acute outer edge. Finally, the first end segment 2 is connected to the second end segment 3 at an interface 7 formed by the first 53 and second 54 interfaces.

The length of the first body portion 51 and the second body portion 52 may be equal as shown in FIG. 6. In another embodiment, the length of the first body portion 51 may be greater than the length of the second body portion 52 as shown in FIG. 3. FIG. 3 also shows that the first interface 53 is a male interface with threads and the second interface 54 is a female interface with counter-threads. A seal 20 is shown in the interface 7 to prevent leakage from the inside to the outside of the multisegment interconnect device 1. While the seal 20 is shown at the bottom of the threaded female interface, the use of any and all fluid tightening technologies, including different placement of the seal, the use of tape, spraying, or lowering of tolerances is contemplated.

In one embodiment shown in FIG. 3, the first acute outer edge on the first end segment 2 is connected to the first locking lips 30 and a back end 31, and the back end 31 is substantially perpendicular to the first body portion. The first locking lips 30 engages plastically within the tube 11 constrained over the lip 30 and nonremovable engagement.

In another embodiment shown in FIG. 8, the multisegment interconnect device 1 with opposing connecting ends includes a second end segment 40 in opposition to the first end segment 2 having a second body portion 41 with a second interface 44, and a second connecting end 42 to connect to a device interface (not shown), wherein the first end segment 2 is connected to the second end segment 40 at an interface formed by the first and second interfaces 53, 44.

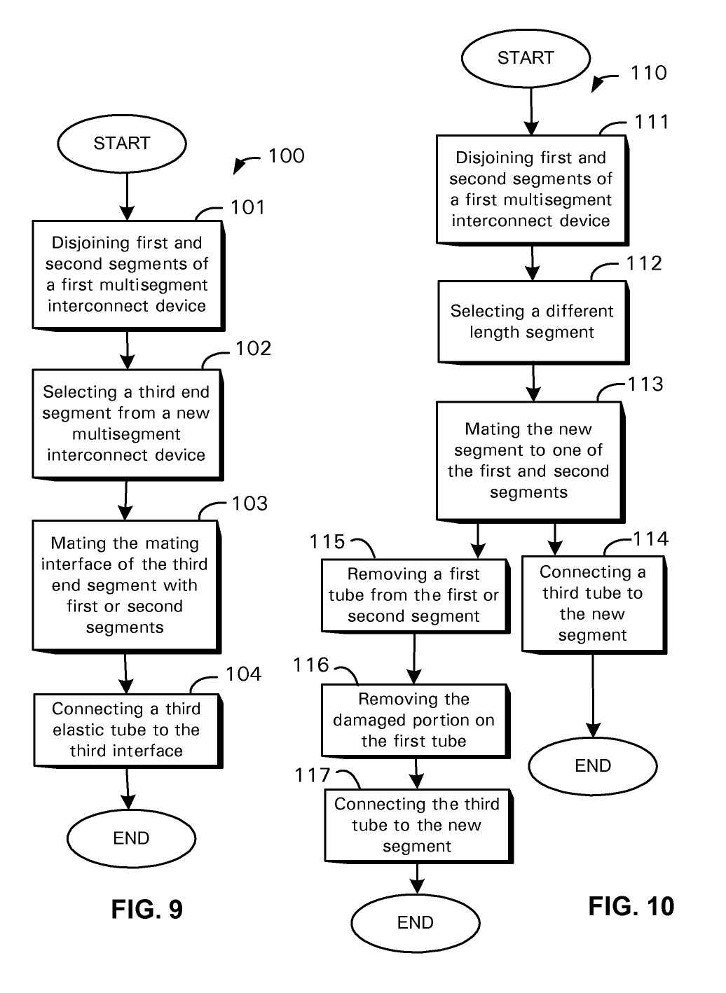

This disclosure also contemplates a method of modifying an elastic tube connection 100 at a connector where elastic tubes are permanently affixed to each of the two opposing connecting ends of a multisegment interconnect device as illustrated schematically in FIG. 9. The device includes a first end segment 2 with a first body portion 51 having a male interface and a first interface 53 to connect to a first elastic tube 11, and a second end segment 3 with a second body portion 52 having a female interface with a second interface 54 to connect to a second elastic tube 12, where the first end segment 2 is connected to the second end segment 3 at an interface 7 formed by mating the male and the female interfaces. The method comprises the steps of disjoining 101 a first and second end segments of a multisegment interconnect device 1 at the male and female interfaces, selecting 102 a third end segment with a mating interface and a third body portion with a third interface to connect a third elastic tube, mating 103 the mating interface of the third end segment to one of the mating interface of either the first and second interfaces, and finally connecting 104 a third elastic tube to the third interface. In one embodiment, the third end segment is of equivalent type and geometry as the first and second end segments 2, 3. During surgery, a physician can use a box with different types of segments to be used interchangeably as part of the multisegment interconnect device 1.

In yet another embodiment shown in FIG. 10, a method of modifying the length of an elastic tube 110 by changing a segment of a multisegment interconnect device 1, the method includes the steps of disjoining 111 a first and second end segments of a multisegment interconnect device 1, selecting 112 a third end segment with a mating interface and a third body portion with a third length with a third interface to connect a third elastic tube, then mating 113 the mating interface of the third end segment to one of the mating interface of either the first and second interfaces, and finally connecting 114 a third elastic tube to the third interface.

In an alternate embodiment, the mating interface of the third end segment is the male interface and is connected to the female interface of the second end segment, and the method 110 comprises further steps of removing 115 the first elastic tube to the first interface using a cutting device, removing 116 any damaged portion off the first elastic tube, and placing 117 the first elastic tube back on the third interface.

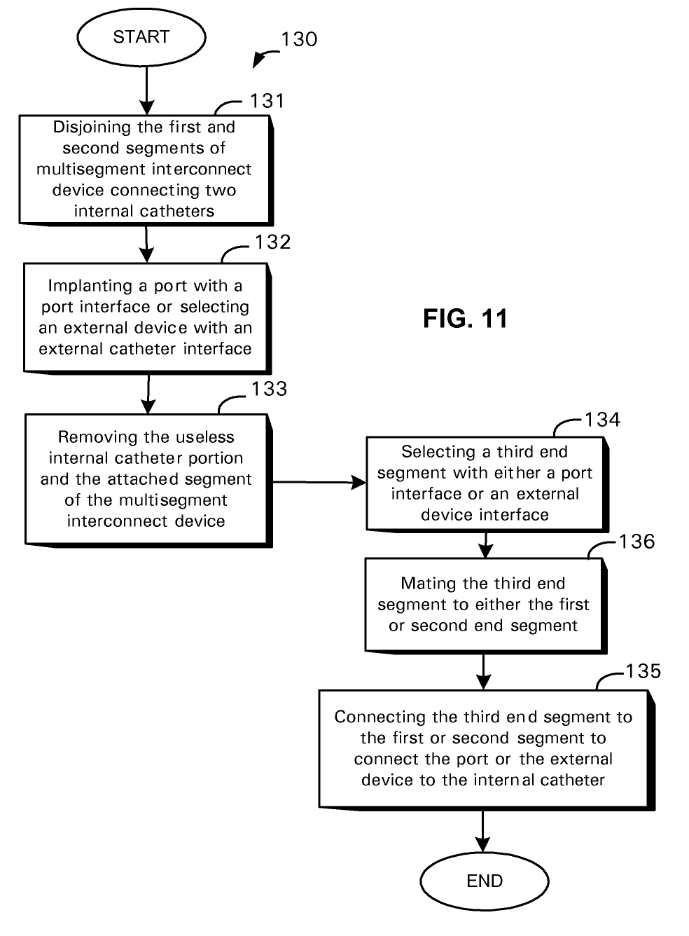

FIG. 11 is a diagrammatic representation of a method for connecting an internal catheter equipped with a multisegment interconnect device 1 to an implanted port or an external catheter. Catheters are types of tubes 11, 12 for the transportation of bodily fluids and can be located in the human body or can be partly located in the body and partly out of the body to connect external devices to the internal fluidic systems of the human body. Internal catheters can be connected to vascular access systems for extracorporeal treatment of blood such as a dialysis machine or to a port as shown in U.S. Pat. No. 5,041,098 filed by one of the inventors, which disclosure is hereby incorporated fully by reference. In fact, patients who begin a session of treatment using external devices often need to migrate to an internal port unless their condition changes. Internal catheters may have to be partly replaced for a plurality of health- and treatment-related reasons, and for this reason, internal catheters in a plurality of segments connected with the device 1 are not uncommon.

Implantable ports are designed to connect via an interface to catheters and may be equipped to receive connectors having an interface. One such interface is shown in FIG. 8 as second end segment 40. External equipment such as dialysis machines have inlets that also require the use of tubes that are then connected to a patient through interfaces such as, for example, element 40 as shown in FIG. 8. While one interface 40 is shown, use of any interface for connecting an external or internal tube of the device 1 to an external device or a port is contemplated. In one embodied method, a treating physician will need to adapt an internal catheter equipped with the device 1 to either a vascular access system or an external catheter for use by an external device.

FIG. 11 shows a method 130 of connecting an internal catheter 11, 12 equipped with a multisegment interconnect device 1 to an implanted port or an external device, the method comprising the steps of disjoining 131 a first and second end segments of a multisegment interconnect device 1 at an interface where the multisegment interconnect device includes the first end segment with a first body portion having a first interface to connect to a first internal catheter and a second end segment with a second body portion having a second interface to connect to a second internal catheter, removing 133 a useless portion of internal catheter and the attached segment of the multi-segment interconnect device, selecting 134 a third end segment with a port interface 40 and having a third interface for connection to the first or second interface, and mating 136 the third interface to the first or second interface, and finally connecting 135 the port interface to a port or an external device. In one alternate embodiment, a port must be implanted 132 in a patient before the connection of the third end segment is connected 135.

It is understood that the preceding detailed description of some examples and embodiments of the present invention may allow numerous changes to the disclosed embodiments in accordance with the disclosure made herein without departing from the spirit or scope of the invention. The preceding description, therefore, is not meant to limit the scope of the invention but to provide sufficient disclosure to one of ordinary skill in the art to practice the invention without undue burden.

What is claimed is:

1. A multisegment interconnect device with opposing connecting ends for the permanent connection of elastic tubes at the connecting ends, the device comprising:
a first end segment with a first body portion with a first interface, and a first connecting end to connect to a first elastic tube having a first end surface adjacent to the first connection surface to guide the first tube over the first end surface to the first connection surface, wherein the first connection surface forms a first acute outer edge with a first locking lip, and the first end surface comprises a first external periphery, and a first conical surface defining a large opening and a small opening wherein the large opening connects to the first acute outer edge; and
a second end segment with a second body portion with a second interface, and a second connecting end to connect to a second elastic tube, having a second end surface adjacent to the second connection surface to guide the second tube over the second end surface to the second connection surface, wherein the second connection surface forms an outer acute edge with a second locking lip, and the second end surface comprises a second external periphery, and a second conical surface defining a large opening and a small opening and wherein the large opening connects to the second acute outer edge,
wherein the first end segment is connected to the second end segment at an interface formed by the first and second interfaces.

2. The multisegment interconnect device of claim 1, wherein a length of the first body portion and the second body portion is equal.

3. The multisegment interconnect device of claim 1, wherein a length of the first body portion is greater than the length of the second body portion.

4. The multisegment interconnect device of claim 1, wherein the first interface is a male interface and the second interface is a female interface.

5. The multisegment interconnect device of claim 1, wherein the interface includes a seal.

6. The multisegment interconnect device of claim 1, wherein the first acute outer edge is connected to the first locking lips and a back end, and wherein the back end is substantially perpendicular to the first body portion.

7. A multisegment interconnect device with opposing connecting ends for the permanent connection of at least one elastic tube at one of the connecting ends, the device comprising:
an end segment with a body portion with a first interface, and a first connecting end to connect to an elastic tube having an end surface adjacent to the connection surface to guide the tube over the first end surface to the first connection surface, wherein the first connection surface forms a first acute outer edge with a first locking lip, and the first end surface comprises a first external periphery, and a first conical surface defining a large opening and a small opening and wherein the large opening connects to the first acute outer edge; and
a second end segment with a second body portion with a second interface, and a second connecting end to connect to a device interface,
and wherein the first end segment is connected to the second end segment at an interface formed by the first and second interfaces.

8. The multisegment interconnect device of claim 7, wherein the device interface is a short end connector.

9. The multisegment interconnect device of claim 7, wherein a length of the first body portion is greater than the length of the second body portion.

10. The multisegment interconnect device of claim 7, wherein the first interface is a male interface and the second interface is a female interface.

11. The multisegment interconnect device of claim 7, wherein the interface includes a seal.

12. The multisegment interconnect device of claim 7, wherein the acute outer edge is connected to the locking lips and a back end, and wherein the back end is substantially perpendicular to the first body portion.

13. A method of modifying an elastic tube connection at a connector where elastic tubes are permanently affixed to each of the two opposing connecting ends of a multisegment interconnect device, the device comprising a first end segment with a first body portion having a male interface and a first interface to connect to a first elastic tube, and a second end segment with a second body portion having a female interface with a second interface to connect to a second elastic tube, wherein the first end segment is connected to the second end segment at an interface formed by mating the male and the female interfaces, the method comprising the steps of:
disjoining a first and second end segments of a multisegment interconnect device at the male and female interfaces;
selecting a third end segment with a mating interface and a third body portion with a third interface to connect a third elastic tube;
mating the mating interface of the third end segment to one of the mating interface of either the first and second interfaces; and
connecting a third elastic tube to the third interface.

14. A method of modifying the length of an elastic tube by changing a segment of a multisegment interconnect device, the method comprising the steps of:
disjoining a first and second end segments of a multisegment interconnect device at a male and a female interface where the multisegment interconnect device includes the first end segment with a first body portion with a first length having the male interface and a first interface to connect to a first elastic tube, and a second end segment with a second body portion with a second length having the female interface with a second interface to connect to a second elastic tube;
selecting a third end segment with a mating interface and a third body portion with a third length with a third interface to connect a third elastic tube;
mating the mating interface of the third end segment to one of the mating interface of either the first and second interfaces; and
connecting a third elastic tube to the third interface.

15. The method of claim 14, wherein the mating interface of the third end segment is the male interface and is connected to the female interface of the second end segment, and wherein the method comprises the steps of removing the first elastic tube to the first interface using a cutting device, removing any damaged portion off the first elastic tube, and placing the first elastic tube back on the third interface.

16. The method of claim 14, wherein the mating interface of the third end segment is the female interface and is connected to the male interface of the first end segment, and wherein the method comprises the steps of removing the second elastic tube to the second interface using a cutting device, removing any damaged portion off the second elastic tube, and placing the second elastic tube back on the third interface.

17. A method of connecting an internal catheter equipped with a multisegment interconnect device to an implanted port, the method comprising the steps of:
disjoining a first and second end segments of a multisegment interconnect device at an interface where the multisegment interconnect device includes the first end segment with a first body portion and a first interface to connect to a first internal catheter, and a second end segment with a second body portion with a second interface to connect to a second internal catheter;
removing a useless portion of internal catheter and the attached segment of the multi-segment interconnect device;
selecting a third end segment with a port interface and having a third interface for connection to the first or second interface;
mating the third interface to either the first or the second interface; and
connecting the port interface to a port.

18. A method of connecting an internal catheter equipped with a multisegment interconnect device to an external catheter, the method comprising the steps of:
disjoining a first and second end segments of a multisegment interconnect device at a male and a female interface where the multisegment interconnect device includes the first end segment with a first body portion and a first interface to connect to a first internal catheter, and a second end segment with a second body portion with a second interface to connect to a second internal catheter;
removing a useless portion of internal catheter and the attached segment of the multi-segment interconnect device;
selecting a third end segment with an external catheter interface and having a third interface for connection to the first or second interface;
mating the third interface to either the first or the second interface; and
connecting the external catheter interface to an external catheter.
